(19) United States
(12) Patent Application Publication
(10) Pub. No.: US 2020/0181187 A1
(43) Pub. Date: Jun. 11, 2020

(54) PROCESS FOR THE PREPARATION OF (S,S)-SECOISOLARICIRESINOL DIGLUCOSIDE AND (R,R)-SECOISOLARICIRESINOL DIGLUCOSIDE

(71) Applicant: LignaMed LLC, Philadelphia, PA (US)

(72) Inventors: Ke LI, Wilmington, DE (US); Thais SIELECKI, Kennett Square, PA (US)

(21) Appl. No.: 16/620,644

(22) PCT Filed: Jun. 11, 2018
(86) PCT No.: PCT/US2018/036859

§ 371 Date: Dec. 9, 2019

Related U.S. Application Data
(60) Provisional application No. 62/518,196, filed on Jun. 12, 2017.

(51) Int. Cl. C07H 15/18; C07H 1/00

(52) U.S. Cl. CPC C07H 15/18; C07H 1/00

(57) ABSTRACT

Provided is a synthesis process for (S,S)-secoisolariciresinol diglucoside and (R,R)-secoisolariciresinol diglucoside.

### FIELD OF THE INVENTION

The field relates to the preparation of (S,S)-secoisolariciresinol diglucoside and (R,R)-secoisolariciresinol diglucoside.

### BACKGROUND OF THE INVENTION

Secoisolariciresinol diglucoside (SDG) is the major lignin in wholegrain flaxseed. SDG is a potent antioxidant and free radical scavenger. Chemically synthesized SDG (S,S and R,R enantiomers) has been demonstrated to share the radioprotective properties of the natural product. See Mishra et al., Radiation Research 182:102-110 (2014) and US Pat. Pub. 2016/0137682.

Synthetic (S,S)- and (R,R)-SDG have been produced by a multistep process that includes a condensation reaction between a diol and a perbenzoyl-protected trichloroacetimidate as a glycosylation donor, under the influence of trimethylsilyl trifluoromethanesulfonate (TMSOTf), to produce a mixture of inseparable blocked diastereomers:

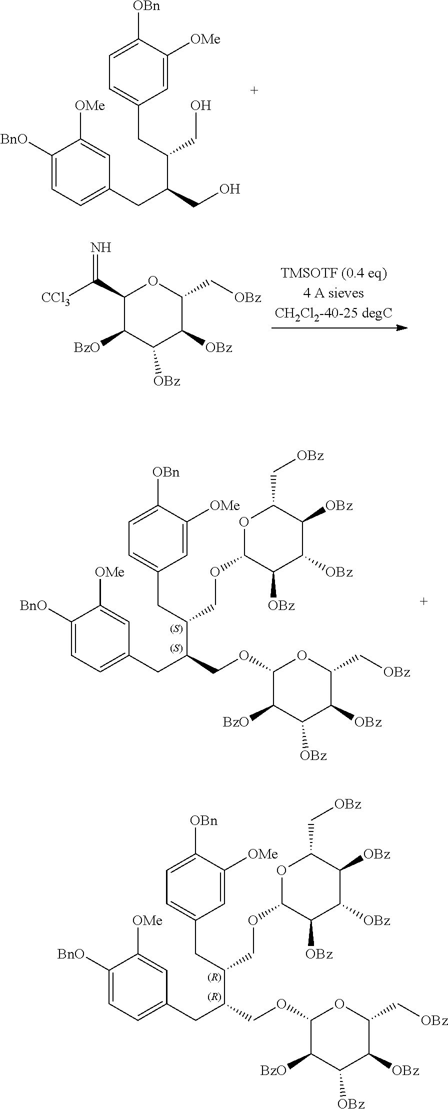

See US Pat. Pub. 2016/0137682. However, the perbenzoyl-protected trichloroacetimidate is unstable and degrades by hydrolysis rapidly after preparation. It must be utilized in the condensation reaction immediately upon preparation. As a result of the ongoing degradation of the trichloroacetimidate during reaction with the diol, the yield of the condensation step, and concomitantly the overall yield of the desired ultimate products (S,S)- and/or (R,R)-SDG, suffers significantly.

What is needed is a process for the production of (S,S)- and (R,R)-SDG that avoids the yield loss attributable to the use of the aforementioned perbenzoyl-protected trichloroacetimidate in the condensation reaction with diol.

### SUMMARY OF THE INVENTION

Provided is a process for preparing a compound of formula ((S,S)-SDG-1), (RR,)-SDG-2) or mixture of compounds of formula ((S,S)-SDG-1) and ((RR,)-SDG-2):

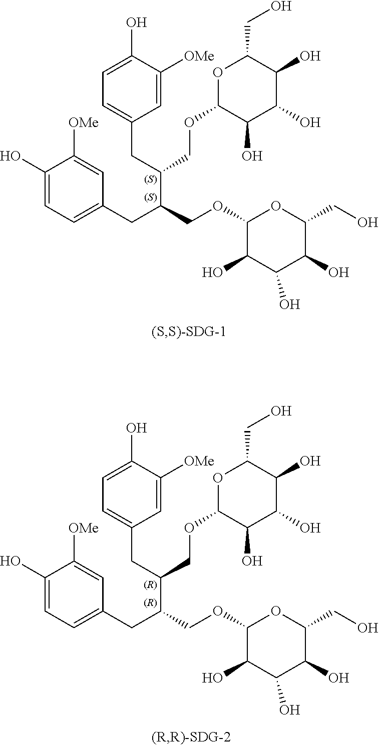

The process comprises:
(a) reacting a compound of formula (1a), (1b) or mixture of compounds of formula (1a) and (1b):

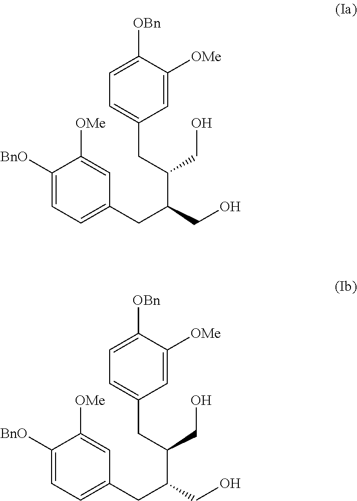 [SMILES: COc1cc(C[C@H](CO)[C@@H](CO)Cc2ccc(OCc3ccccc3)c(OC)c2)ccc1OCc1ccccc1 ; COc1cc(C[C@@H](CO)[C@H](CO)Cc2ccc(OCc3ccccc3)c(OC)c2)ccc1OCc1ccccc1]

with a compound of formula (2):

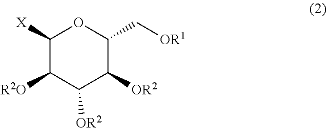

wherein X is a halogen, and R¹ and each R² are independently a protective group, to prepare a mixture of compounds of formula ((S,S)-3) and ((R,R)-4):

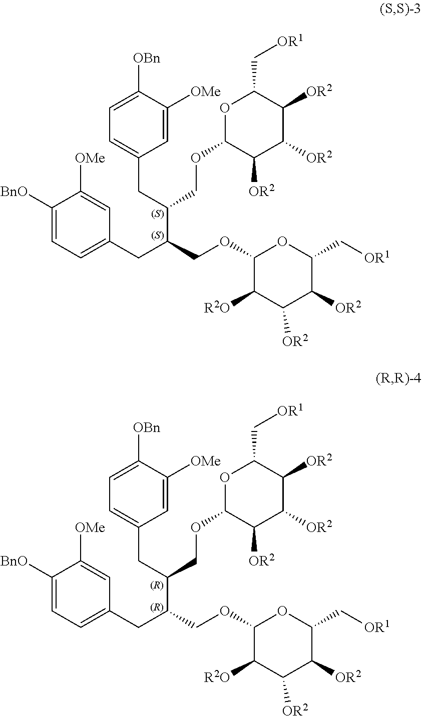

(b) cleaving benzyl ethers of the compounds of formula ((S,S)-3) and ((R,R)-4) to provide a mixture of compounds of formula ((S,S)-5) and ((R,R)-6):

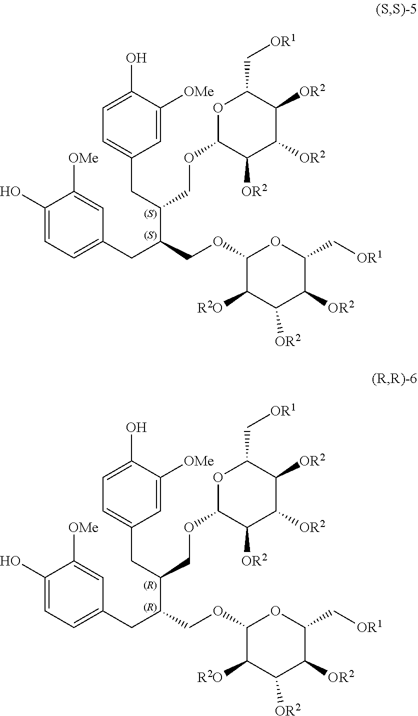

(c) optionally separating the mixture of compounds of formula ((S,S)-5) and ((R,R)-6) to provide a compound of formula ((S,S)-5) or a compound of formula ((R,R)-6); and
(d) deprotecting the compound of formula ((S,S)-5), the compound of formula ((R,R)-6) or a mixture of compounds of formula ((S,S)-5) and ((R,R)-6), to provide a compound of formula ((S,S)-SDG-1), ((RR,)-SDG-2) or mixture of compounds of formula ((S,S)-SDG-1) and ((RR,)-SDG-2).

Also provided is a process for preparing a mixture of compounds of formula ((S,S)-3 and ((R,R)-4):

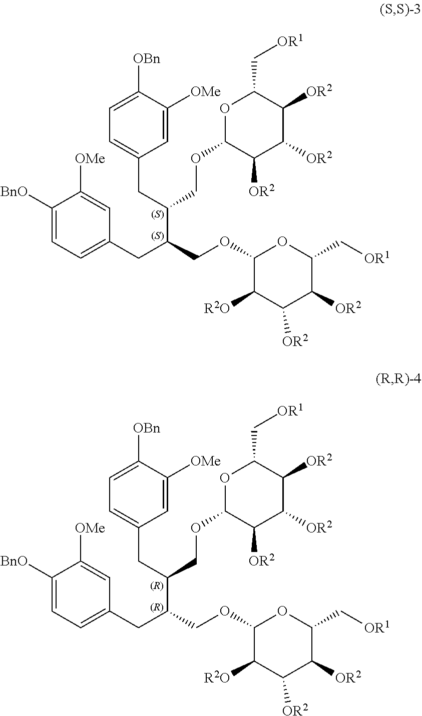

wherein R¹ and each R² are independently a protective group.

The process comprises:
(a) reacting a compound of formula (1a), (1b), or mixture of compounds of formula (1a) and (1b):

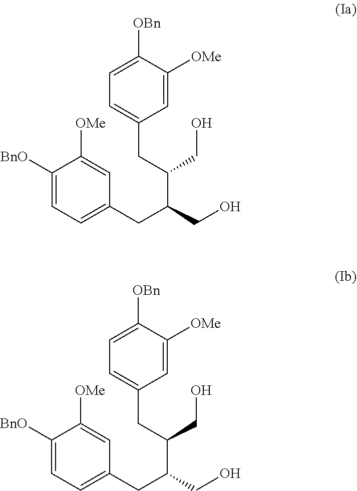 [SMILES: COc1cc(C[C@H](CO)[C@@H](CO)Cc2ccc(OCc3ccccc3)c(OC)c2)ccc1OCc1ccccc1 ; COc1cc(C[C@@H](CO)[C@H](CO)Cc2ccc(OCc3ccccc3)c(OC)c2)ccc1OCc1ccccc1]

with a compound of formula (2):

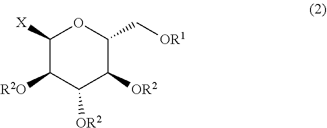

wherein X is a halogen, and R¹ and each R² are independently a protective group, to prepare a mixture of compounds of formula ((S,S)-3) and ((R,R)-4).

Also provided is a process for preparing a compound of formula ((S,S)-5), ((R,R)-6) or a mixture of compounds of formula ((S,S)-5) and ((R,R)-6):

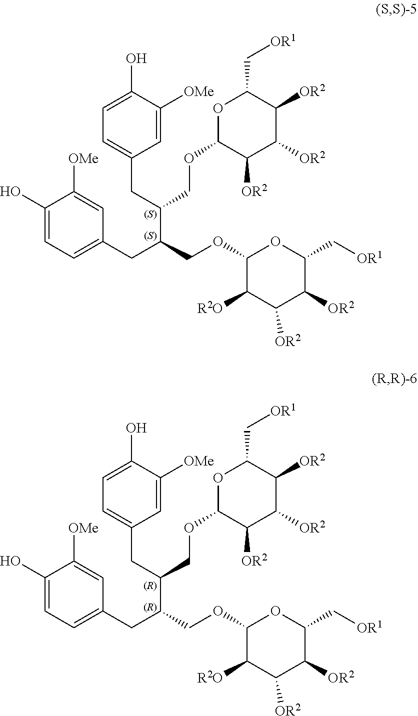

wherein R¹ and each R² are independently a protective group.

The process comprises:
(a) reacting a compound of formula (1a) (1b), or mixture of compounds of formula (1a) and (1b):

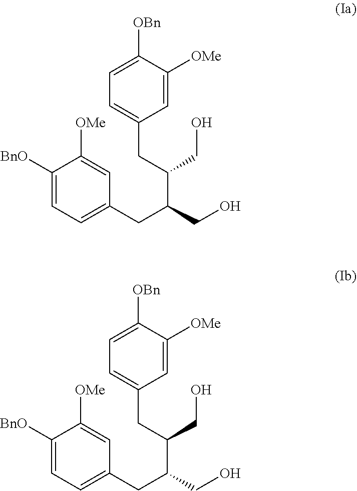 [SMILES: COc1cc(C[C@H](CO)[C@@H](CO)Cc2ccc(OCc3ccccc3)c(OC)c2)ccc1OCc1ccccc1 ; COc1cc(C[C@@H](CO)[C@H](CO)Cc2ccc(OCc3ccccc3)c(OC)c2)ccc1OCc1ccccc1]

with a compound of formula (2)

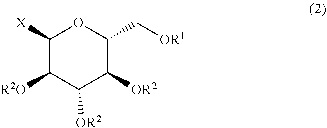

wherein X is a halogen, and R¹ and each R² are independently a protective group, to prepare a mixture of compounds of formula ((S,S)-3) and ((R,R)-4):

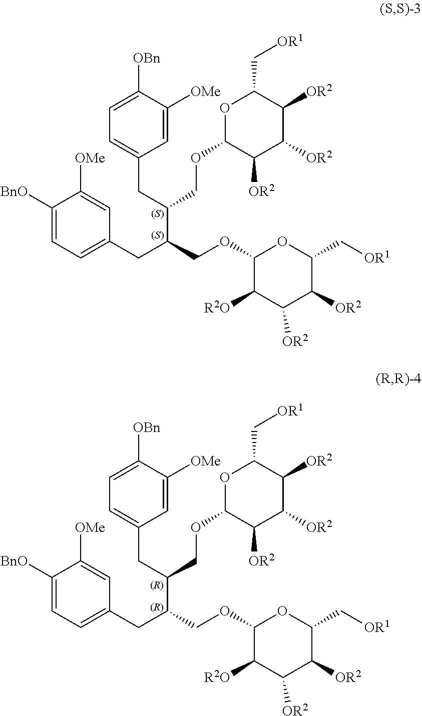

(b) cleaving benzyl ethers of the mixture of compounds of formula ((S,S)-3) and ((R,R)-4) to provide a mixture of compounds of formula ((S,S)-5) and ((R,R)-6).

In certain embodiments or any of the aforementioned processes, the compound of formula (1a) is reacted in step (a),

In certain embodiments of any of the aforementioned processes, the compound of formula (1b) is reacted in step (a).

In certain embodiments of any of the aforementioned processes, a mixture of the compound of formula (1a) and the compound of formula (1b) is reacted in step (a).

In certain embodiments of any of the aforementioned processes, R¹ and each R² are independently selected from acetyl and benzoyl. In certain embodiments, R¹ and each R² are benzoyl.

In certain embodiments of any of the aforementioned processes, X in the compound of formula (2) is bromine. In certain embodiments of any of the aforementioned processes, X in the compound of formula (2) is bromine and R¹ and each R² are benzoyl.

In certain embodiments of any of the aforementioned processes, step (a) is carried in the presence of a halide ion acceptor. The halide ion acceptor may for example comprise, in any of the embodiments of the aforementioned processes, a heavy metal salt. In certain embodiments, the halide ion acceptor is a salt of silver or mercury. In certain embodiments, the salt of silver or mercury is selected from the group consisting of CAgF₃O₃, Ag₂O, Ag₂CO₃, AgO₂CCH₃, AgClO₄, Hg(CN)₂, HgBr₂, and combinations thereof

In certain embodiments of any of the aforementioned processes, step (a) is carried out in the presence of activated molecular sieves.

In certain embodiments, the mixture of compounds of formula ((S,S)-5) and ((R,R)-6) obtained in step (b) is separated to provide a compound of formula ((S,S)-5) or a compound of formula ((R,R)-6), and at least one of them is deprotected in step (d).

As envisioned in the present invention with respect to the disclosed compositions of matter and methods, in one aspect the embodiments of the invention comprise the components and/or steps disclosed herein. In another aspect, the embodiments of the invention consist essentially of the components and/or steps disclosed herein. In yet another aspect, the embodiments of the invention consist of the components and/or steps disclosed herein.

Any open valence appearing on a carbon, oxygen or nitrogen atom in the structures herein indicates the presence of a hydrogen atom.

### DEFINITIONS

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +/−20% or +/−10%, more preferably +/−5%, even more preferably +/−1%, and still more preferably +/−0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

“Bn” means the benzyl group, C₆H₅CH₂—.

“Bz” means the benzoyl group, C₆H₅C(O)—.

“Me” means the methyl group, —CH₃.

“Ac” means the acyl group.

By “substantially pure” is meant compositions containing, by weight, at least about 80 of the desired diastereomer.

### BRIEF DESCRIPTION OF THE FIGURES

FIG. 1 depicts a synthesis scheme according to the present invention.

FIG. 2 is a ¹³C NMR spectrum of a mixture of (S,S)-SDG-1 and (R,R)-SDG-2 obtained by the method of the present invention.

FIG. 3A is an ¹H NMR spectrum of a mixture of (S,S)-SDG-1 and (R,R)-SDG-2 obtained by the method of the present invention.

FIG. 3B shows the conditions of the operation of the instrument yielding the ¹H NMR spectrum of FIG. 3A.

FIG. 4 depicts an alternative synthesis scheme according to the present invention.

### DETAILED DESCRIPTION OF THE INVENTION

A process is provided for preparing a compound of formula ((S,S)-SDG-1), ((RR,)-SDG-2) or mixture of compounds of formula ((S,S)-SDG-1) and ((RR,)-SDG-2):

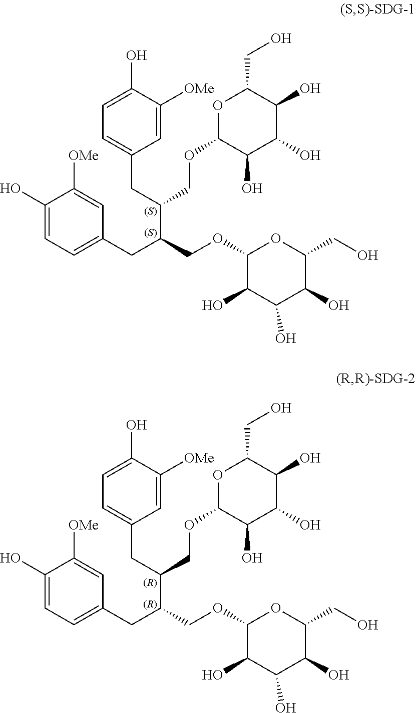

The process comprises:
(a) reacting a compound of formula (1a), (1b), or mixture of (1a) and (1b):

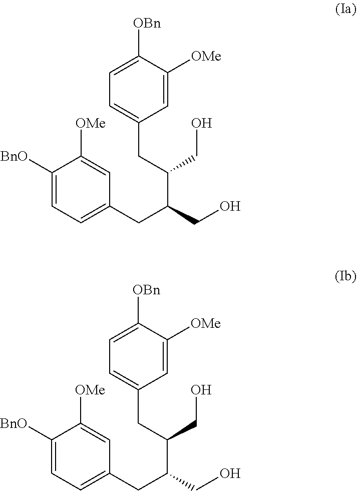 [SMILES: COc1cc(C[C@H](CO)[C@@H](CO)Cc2ccc(OCc3ccccc3)c(OC)c2)ccc1OCc1ccccc1 ; COc1cc(C[C@@H](CO)[C@H](CO)Cc2ccc(OCc3ccccc3)c(OC)c2)ccc1OCc1ccccc1]

with a compound of formula (2):

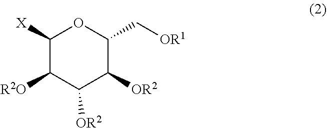

wherein X is a halogen, and R¹ and each R² are independently a protective group, to prepare a mixture of compounds of formula ((S,S)-3) and ((R,R)-4):

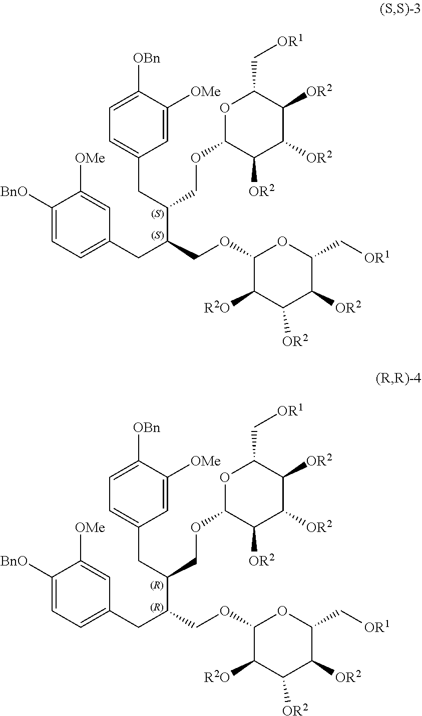

(b) cleaving benzyl ethers of the mixture of compounds of formula ((S,S)-3) and ((R,R)-4) to provide a mixture of compounds of formula ((S,S)-5) and ((R,R)-6):

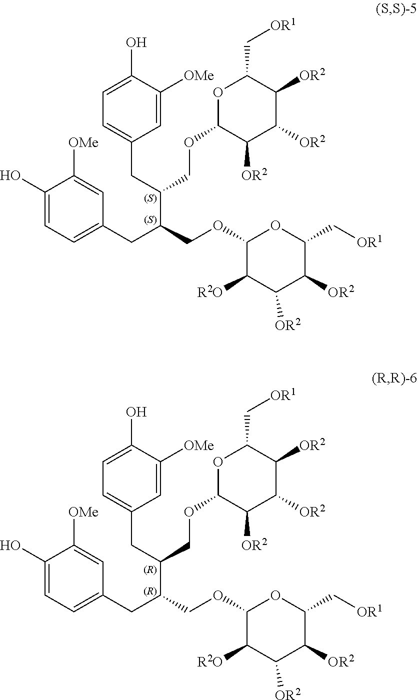

(c) optionally separating the mixture of compounds of formula ((S,S)-5) and formula ((R,R)-6), to provide a compound of formula ((S,S)-5) or a compound of formula ((R,R)-6); and
(d) deprotecting the compound of formula ((S,S)-5), the compound of formula ((R,R)-6) or mixture of compounds of formula ((S,S)-5) and ((R,R)-6) to provide a compound of formula ((S,S)-SDG-1), a compound of formula ((RR,)-SDG-2) or mixture of compounds of formula ((S,S)-SDG-1) and ((RR,)-SDG-2).

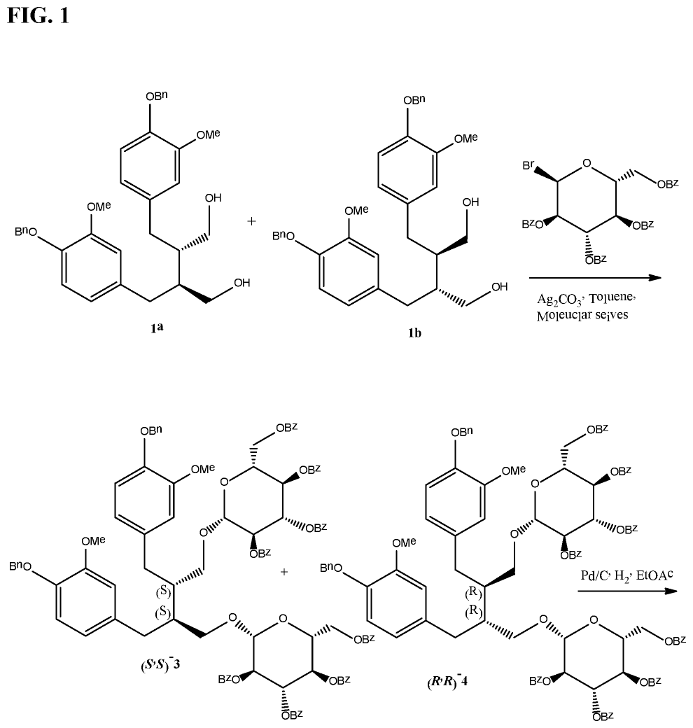

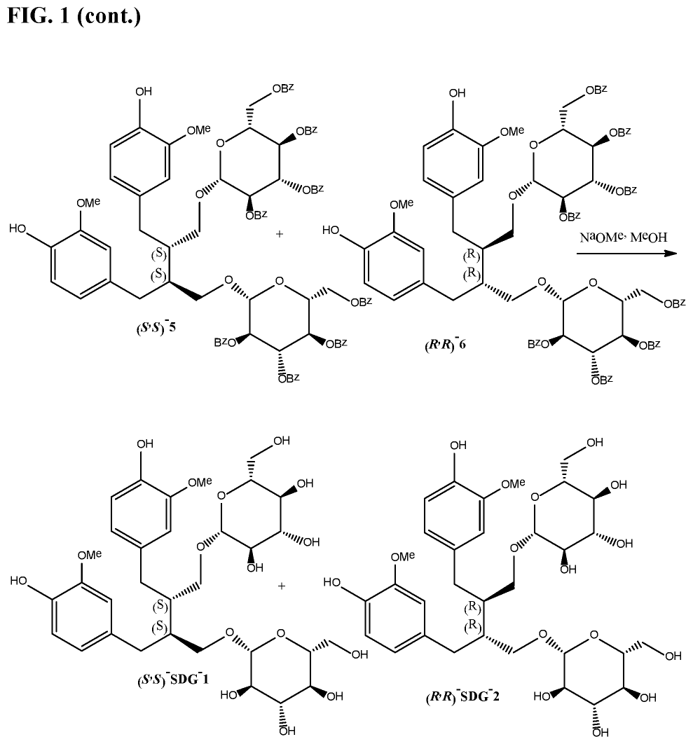

FIG. 1 illustrates a reaction scheme according to the present invention where the compound of formula 2 is α-D-glucopyranosyl bromide tetraheozoate (formula 2, X=Br; R¹=Bz; R²=Bz).

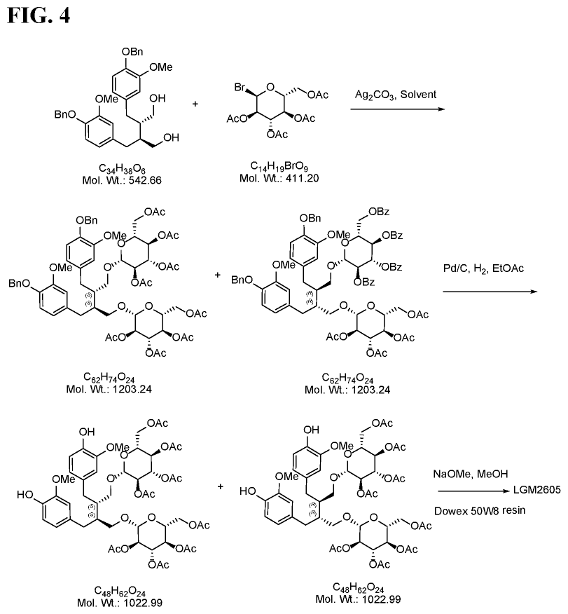

FIG. 4 illustrates an alternative reaction scheme according to the present invention utilizing a different compound of formula 2 (X=Br; R¹=Ac; R²=Ac). The resulting “LGM2605” is a mixture of compounds of formula ((S,S)-SDG-1) and ((R,R)-SDG-2). In step (d). purification is performed utilizing a Dowex 50w8 resin column.

In some embodiments, the reaction between the compound of formula (1a) or (1b), or mixture of compounds of formula (1a) and (1b), with a compound of formula (2) (hereinafter “coupling reaction”, “coupling step” or “glycosylation reaction”) is carried out in the presence of a halide ion acceptor that promotes the reaction. In some embodiments, the halide ion acceptor is a heavy metal salt. In some embodiments, the heavy metal salt is a silver salt, such as silver trifluoromethanesulfonate (CAgF₃O₃ or “AgOTf”), Ag₂O, Ag₂CO₃, silver acetate (AgO₂CCH₃), AgClO₄, or combinations thereof. In some embodiments, the heavy metal salt is a mercury salt, such as Hg(CN)₂, HgBr₂, or a combination thereof. Ag2CO₃ is a preferred halide ion acceptor from the standpoint of efficacy and cost. For the preparation of O-glycosides via coupling reactions catalyzed by heavy metal halide ion acceptors, see Brito-Arias, Synthesis and Characterization of Glycosides, Chapter 2, “O-Glycoside Formation”, p. 68-136, Springer US, 2007.

The coupling reaction may be carried out in a solvent. Suitable solvents include toluene, dichloromethane, diethylether, tetrahydrofuran, carbon tetrachloride, and the like.

Activated molecular sieves may be optionally added to the reaction mixture before addition of halide ion acceptor in order to absorb adventitious water and hydrogen halide liberated from the coupling reaction. Molecular sieves may be activated for use by known methods of drying. Drying may proceed, for example, by heating molecular sieves in a dry vessel to 120° C. overnight under vacuum. The activated molecular sieves may take an appropriate form, e.g., bead or powders. A particularly useful sieve size is about 3 angstroms, or about 4 angstroms.

The diol is enantiomeric, and may exist as either (2S,3S)-2,3-bis(4-(benzyloxy)-3-methoxybenzyl)butan-1,4-diol (formula (1a)) or (2R, 3R)-2,3-bis(4-(benzyloxy)-3-methoxybenzyl)butan-1,4-diol (formula (1b)). Diastereomerically pure compound of formula (1b) may be prepared according to known methods. See, for example, the synthesis from vanillin set forth in US Pat. Pub. 2016/0137682, Examples 1-5, and Mishra et al., Bioorg Med Chem Lett 2013, 23(19):5325-8. The entire disclosures of the aforesaid documents are incorporated herein by reference.

The compound of formula 2 comprises protective groups R¹ and R², R¹ and each occurrence of R² may be independently selected. The protective groups comprise any suitable groups for protecting the functional group —OH. Appropriate protecting groups for hydroxyl and phenol groups, and methods for their removal, are widely known in the art and include, for example, those described in Chapters 2 and 3 of Greene's Protective Grow In Organic Synthesis, 4th Ed., John Wiley & Sons, Inc., 2007, by Peter G. M. Wuts, Theodora W. Greene.

Protective groups include, for example, aliphatic acyl groups and aromatic acyl groups, which may be optionally substituted. Examples of acyl groups include (C₁-C₇)acyl groups (e.g. alkanolyl or benzoyl) which may be optionally substituted. Substituents may include, for example, (C₁-C₃)alkoxy, halogen, (C₁-C₃)alkanoyloxy, nitro or hydroxyl. Multiple substitutions on the acyl groups are possible.

Specific examples of acyl groups include formyl, acetyl, ethoxyacetyl, fluoroacetyl, difluoroacetyl, trifluoroacetyl, chloroacetyl, dichloroacetyl, trichloroacetyl, bromoacetyl, dibromoacetyl, tribromoacetyl, propionyl, 2-chloropropionyi, 3-chloropropionyl, butyryl, 2-chlorobutyryl, 3-chlorobutyryl, 4-chlorobutyryl, 2-methylbutyryl, 2-ethylbutyryl, valeryl, 2-methylvaleryl, 4-methylvaleryl, hexanoyl, isobutyryl, isovaleryl, pivaloyl, benzoyl, o-chlorobenzoyl, m-chlorobenzoyl, p-chlorobenzoyl, o-hydroxybenzoyl, m-hydroxybenzoyl, p-hydroxybenzoyl, o-acetoxybenzoyl, o-methoxybenzoyl, m-methoxybenzoyl, p-methoxybenzoyl, p-nitrobenzoyl, and the like.

Protective groups may include tri-substituted silyl groups. Tri-substituted silyl groups include, silyl groups substituted with 3 substituent groups selected from, for example, phenyl and (C₁-C₄)alkyl. Specific examples of such silyl groups include trimethylsilyl, triphenylsilyl, triethylsilyl, triisopropylsilyl, t-butyldimethylsilyl, t-butyldiphenylsilyl, and the like.

Protective groups further include alkoxyalkyl groups, such as tetrahydropyranyl, methoxymethyl, methoxyethoxymethyl and 1-ethoxyethyl. The carbon chain lengths of the alkoxy and alkyl residues are preferable from C₁ to C₆.

Protective groups further include saturated or unsaturated hydrocarbyloxycarbonyl groups which may be optionally substituted. Examples include (C₁-C₄)alkoxycarbonyl which may be substituted with a halogen atom or atoms, and allyloxycarbonyl. Examples of optionally substituted (C₁-C₄)alkoxycarbonyi groups include methoxycarbonyl, ethoxycarbonyl and 2,2,2-trichloroethoxycarbonyl.

Protective groups further include optionally substituted benzyl, particularly (C₁-C₆)alkoxy-substituted benzyl. Examples include benzyl and p-methoxybenzyl.

Protective groups further include groups having tertiary carbon substituted with an aryl or alkyl group. Examples include a group having a tertiary carbon substituted with three substituent groups selected from an aryl group (e.g. phenyl) and a (C₁-C₃)alkyl. Specific examples include trityl and t-butyl.

The aforementioned lists of protective groups are intended to be exemplary, and not limiting, Preferred are acyl groups, most particularly, acetyl and benzoyl.

Compounds of formula 2 where R² and R² are benzoyl are commercially available, or may be prepared according to known methods. See, for example, Ravindranathan et al., J. Carbohydr. Chem. 1990, 9, 777-781; Dowlut et al., J. Org. Chem. 2005, 70, 9809, describing the preparation of α-D-glucopyranosyl bromide tetrabenzoate (formula 2, X=Br). The latter compound is also available commercially (Sigma-Aldrich, St. Louis, Mo.) and is identified by CAS number 14218-11-2. Preferred compounds of formula 2 are the compounds where X is bromine, chlorine or iodine, with bromine being preferred. Further preferred are compounds where X is bromine, chlorine or iodine, and R¹ and R² are selected from acetyl and benzoyl.

Unlike the perbenzoyl-protected trichloroacetimidate of US Pat. Pub. 2016/0137682, the compounds of formula 2 are stable for prolonged periods. In particular, α-D-glucopyranosyl bromide tetrabenzoate has been observed to be stable upon storage at room temperature for up to at least about 1 year. A further advantage of the formula 2 compound resides in the ability to halt and restart its coupling reaction with diol (1a/1b), which reaction may be conducted throughout at about room temperature. The reaction is heterogeneous in nature, which allows the reaction to be readily halted by removal of the heterogeneous metal salts from the reaction solution. The corresponding coupling reaction with the perbenzoyl-protected trichloroacetimidate as glycosylation donor is initiated at −40° C. and allowed to warm overnight to 25° C. Once initiated, the coupling reaction of the perbenzoyl-protected trichloroacetimidate cannot easily be halted, since it is a homogeneous reaction. The reaction may only be stopped by quenching.

The coupling reaction with the formula 2. compound as the glycosylation donor is readily scalable. The coupling reaction with the perbenzoyl-protected trichloroacetimidate is cryogenic in nature. The reduced temperature requires specialized equipment which is expensive to operate, thereby limiting scalability. Most significantly, the enhanced stability of the formula 2 compound results in a coupling step yield, and concomitantly an overall (S,S)-/(R,R)-SDG yield, that is at least about double the yield obtainable with the perbenzoyl-protected trichloroacetimidate.

The perbenzoyl-protected trichloroacetimidate utilized in the process of US Pat. Pub. 2016/0137682 is typically prepared by selective hydrolysis of the formula 2 bromide to the corresponding alcohol (OH for Br), followed by conversion of the hydroxyl compound to the trichloroacetimidate. Direct utilization of the formula 2 bromide as the glycosylation donor in the coupling reaction of the process of the present invention, an expedient not contemplated in the process described in US Pat. Pub. 2016/0137682, results in further enhanced product yield, reagent cost saving and time by eliminating the two steps required for conversion of the bromide to the trichloroacetimidate.

The product of the coupling reaction according to the present invention is a diastereomeric mixture comprising compounds of formulae ((S,S)-3) and ((R,R)-4). Cleaving benzyl ether groups (-OBn) of the coupling reaction product results in a diastereomeric mixture of compound of formulae ((S,S)-5) and ((R,R)-6). In some embodiments, the cleavage of the benzyl ether groups is carried out in in the presence of H₂ and a catalyst comprising palladium on activated carbon (Pd/C). The weight percent loading of Pd may comprise, for example, 10 wt. %. Other loading levels are possible. The hydrogenation reaction may be conducted under elevated pressure, e.g., 75 psi, or at atmospheric pressure, The hydrogenation reaction proceeds at room temperature. The slurry reaction product is filtered, and the solid washed with an appropriate solvent (e.g., ethyl acetate), and concentrated.

According to Step (c), the ((S,S)-5)/((R,R)-6) diastereomeric mixture may be optionally separated to provide substantially purified substantially purified ((S,S)-5) and/or ((R,R)-6) The substantially purified diastereomer may comprise, in preferred embodiments, at least about 85%, at least about 90%, at least about 95%, at least about 97%, at least about 98%, at least about 99%, at least about 99.5% or at least about 99.9% of the desired diastereomer, by weight. Diastereomer separation may be achieved, for example, by thin layer chromatography. Representative thin layer chromatography conditions may comprise, for example: silica, 2 mm, multiple plates, 7:20 EtOAc:hexanes, >10 elution runs. See, US Pat. Pub. 2016/0137682, Example 7.

Step (d) of the process comprises deprotecting the optionally substantially purified compound of formula ((S,S)-5) and/or ((R,R)-6), or a mixture of compounds of formula ((S,S)-5) and ((R,R)-6), to provide substantially purified compound of formula ((S9-SDG-1) and/or ((RR,)-SDG-2), or mixture of compounds of formula ((S,S)-SDG-1) and ((RR,)-SDG-2.

Deprotection proceeds by hydrolysis of O—R¹ and O—R² moieties on ((S,S)-5)/((R,R)-6) to the corresponding alcohol by treatment with an appropriate hydrolysis reagent(s). The selection of the appropriate hydrolysis reagent based upon the nature of the protective group is described, for example, in Greene's Protective Groups in Organic Synthesis, 4th Ed., John Wiley & Sons, Inc., 2007, by Peter G. M. Wuts, Theodora W. Greene, the entire disclosure of which is incorporated herein by reference. In certain embodiments, such as when the protective group is an acyl group, the hydrolysis reagent is a solution of NaOMe in MeOH. The hydrolysis reaction upon completion may be quenched with e.g., HCl in isopropyl alcohol solution, and the product solution concentrated to dryness. The resulting crude solid product may be purified by, for example, column chromatography.

Substantially pure final SDG product may be obtained by separation of the diastereomeric diglucoside intermediates ((S,S)-5) and ((R,R)-6), followed by deprotection of the separated intermediates to provide the corresponding separate ((S,S-SDG-1) and ((RR,)-SDG-2 compounds.

The product resulting from the method of the invention may be amorphous or crystalline.

The process of the invention is thus capable of yielding substantially pure ((S,S)-SDG-1) or ((RR,)-SDG-2), as desired. By “substantially pure” is meant compositions containing, by weight, at least about 80%, at least about at least about 80%, at least about 85%, at least about 90%, at least about 95%, at least about 97%, at least about 98%, at least about 99%, at least about 99.5% or at least about 99.9% of the desired diastereomer. In some embodiments, the substantially purified diastereomer may comprise at least about 85%, at least about 90%, at least about 95%, at least about 97%, at least about 98%, at least about 99%, at least about 99.5% or at least about 99.9% of the desired diastereomer, by weight.

The practice of the invention is illustrated by the following non-limiting examples. The skilled person skilled in the art will appreciate that it may be necessary to vary the procedures for any given embodiment of the invention. For example, reaction monitoring, such as by using thin layer chromatography, or HPLC may be used to determine the optimum reaction time. Products may be purified by conventional techniques that will vary, for example, according to the amount of side products produced and the physical properties of the compounds. On a laboratory scale, recrystallization from a suitable solvent, column chromatography, normal or reverse phase HPLC, or distillation are all techniques which may be useful. The person skilled in the art will appreciate how to vary the reaction conditions to synthesize any given compound within the scope of the invention without undue experimentation. See, e.g., Vogel's Textbook of Practical Organic Chemistry, by A. I. Vogel, et al., Experimental Organic Chemistry: Standard and Microscale, by L. M. Harwood et al. (2nd Ed., Blackwell Scientific Publications, 1998), and Advanced Practical Organic Chemistry, by J. Leonard, et al. (2nd Edition, CRC Press 1994),

### EXAMPLES

### Example 1

Synthesis of ((S,S)-3) and ((R,R)-4)

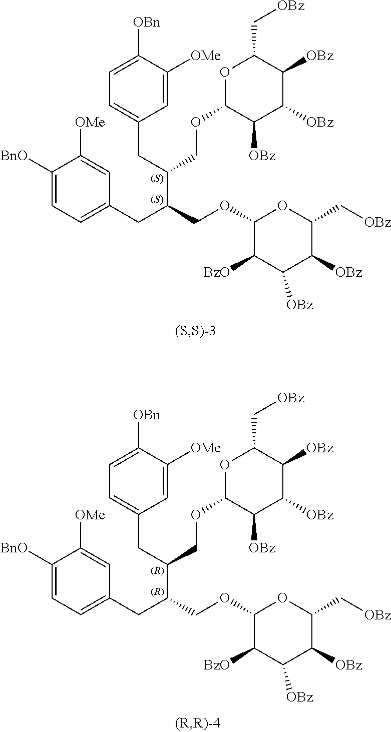

A mixture of the diols (1a) and (1b) (217 g, 0.4 mol) was dried anotropically with toluene (700 mL) before being combined with the α-D-glucopyranosyl bromide tetrabenzoate (791.4 g, 1.2 mol) in round bottom with another 6000 mL of toluene. Molecular sieves (200 g) were added to the slurry before silver carbonate (220.6 g, 0.8 mol) as promoter was added to the mixture in the dark at room temperature. The reaction was kept under nitrogen until HPLC analysis indicated the reaction was complete. The slurry then was filtered through a pad of Celite® diatomaceous earth to remove all solids, The solids were washed thoroughly with ethyl acetate. The combined reaction product solution was concentrated, dissolved in dichromomethane and purified by column chromatography. The purity of each fraction was checked by TLC and HPLC. The fractions with purity>95% were combined and concentrated to give 500 g of a 1:1 mixture of (S,S)-3 and (R,R)-4 as a white foamy product.

### Example 2

Synthesis of ((S,S)-5) and ((R,R-6)

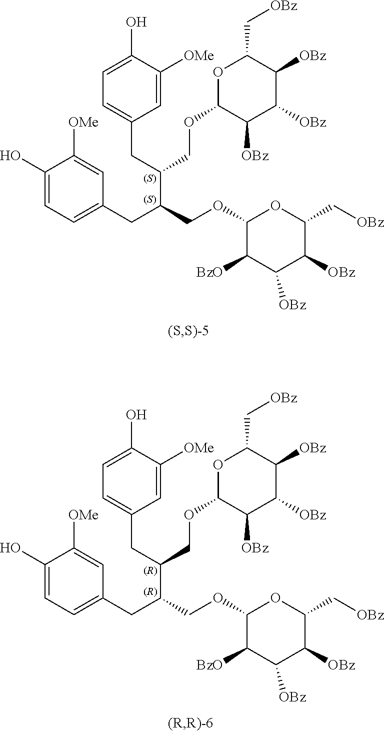

A stirred solution of a 1:1 mixture of (S,S)-3 and (R,R)-4 (530.0 g, 0.31 mol) in EtOAc (2000 mL) was saturated with nitrogen atmosphere by briefly exposing to vacuum and backfilling with nitrogen several times. Palladium on carbon (10% Pd by weight, 30.0 g) was added and the solution was saturated with H₂ by vacuum/backfill H₂. The whole reaction mixture was stirred for 18-36 h under H₂ atmosphere at ambient pressure in a round bottom flask. The reaction mixture was monitored by TLC. After completion of reaction, the solution was put under nitrogen atmosphere, filtered through a pad of Celite® diatomaceous earth and washed with EtOAc (500 mL) and CH₂Cl₂ (500 mL), and the filtrate concentrated to yield 400 g of a 1:1 mixture of (S,S)-5 and (R,R)-6 as a white solid. The solid was used in the next Example without further purification.

### Example 3

Synthesis of ((S,S)-SDG-1 and ((R,R)-SDG-2)

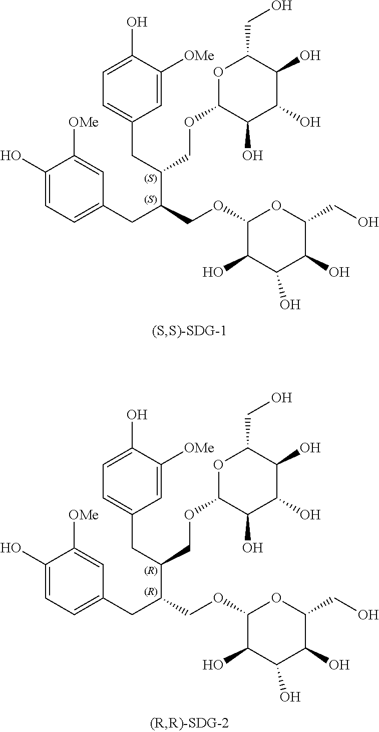

To a stirred solution of the 1:1 mixture of (S,S)-5 and (R,R)-6 from Example 2 (400.0 g, 0.26 mol) in MeOH (2500 mL) was added a solution of 30% NaOMe in MeOH (140 mL) at 5° C. under N₂ atmosphere through a dropping funnel for 30 min. After completion of the addition, the solution was stirred for 40 h at room temperature. The reaction was cooled and quenched with HCl in isopropyl alcohol (IPA) to reach pH ˜5 (˜4 Molar HCl in IPA), After 10 minutes, volatiles were removed under reduced pressure. The residual was dissolved in MeOH (1000 mL) and cooled in the refrigerator overnight. The solid was removed by filtration. The solution was then absorbed onto silica gel and dried under vacuum. The obtained silica mixture was loaded onto a packed column. The product was purified by column chromatography (silica, 60-120 mesh) using 10-20% MeOH/dichloromethane. The product was checked by TLC and HPLC. Those fractions with >97% purity were combined and concentrated to give ˜100 g of 1:1 mixture of (S,S)-SDG-1 and (R,R)-SDG-2 as a white solid.

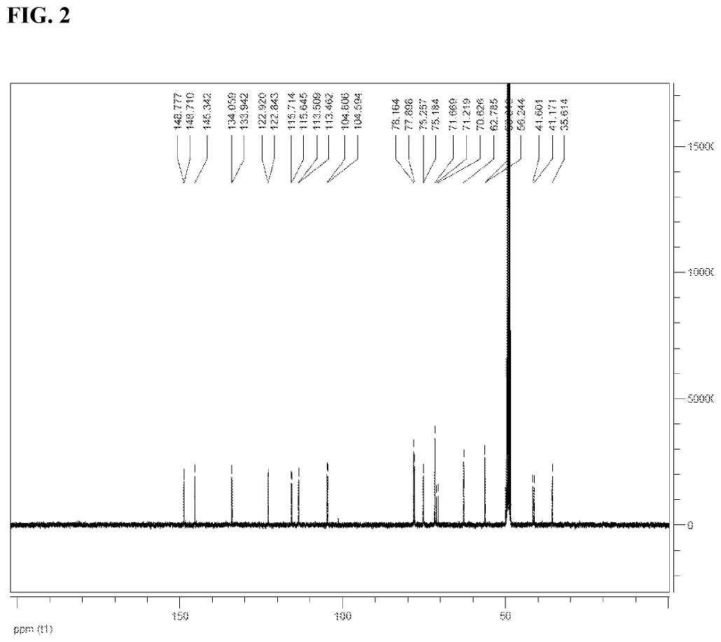

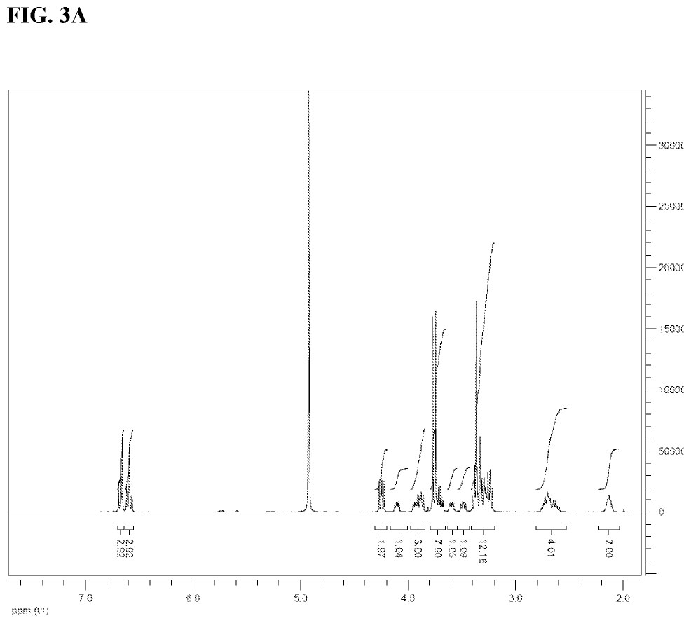

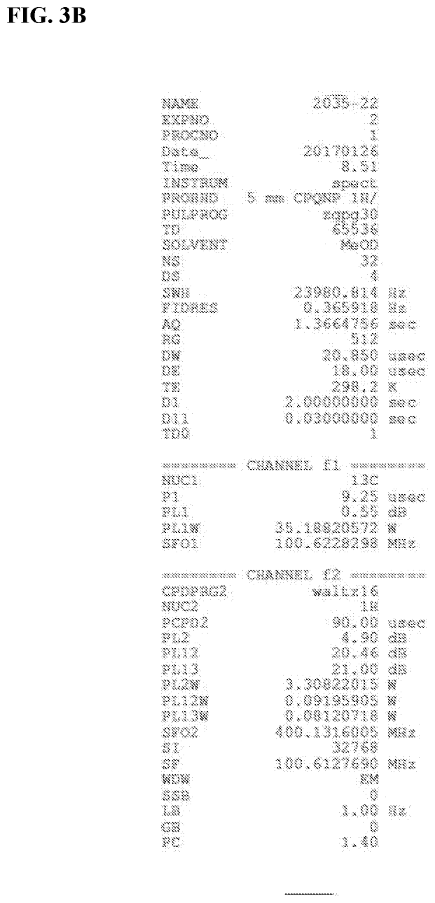

Analytical: Two batches of the aforementioned SDG product were combined and homogenized in methanol solution on a rotary evaporator and then the solvent was removed to again give a white solid. This final product was dried in vacuum oven at 40° C. to reach constant weight, The product was analyzed by ¹H and ¹³C NMR, and LC-MS to confirm the identity of the product as a mixture of (S,S)-SDG-1 and (R,R)-SDG-2. The ¹³C NMR spectrum of the final product (100 MHz; MeOD) is shown in FIG. 2. The ¹ H NMR spectrum of the final product (400 MHz; MeOD) is shown in FIG. 3A. FIG. 3B shows the conditions of the operation of the instrument yielding the ¹H NMR spectrum of FIG. 3A.

Overall Yield for Examples 1 through 3: A 43% overall yield of a (S,S)-SDG-1 and (R,R)-SDG-2 product mixture, based on the amount of starting diol mixture (1a)/(1b), was obtained.

All references disclosed herein are incorporated by reference. One skilled in the art will readily appreciate that the present invention is well adapted to carry out the objects and obtain the ends and advantages mentioned, as well as those inherent therein. The present invention may be embodied in other specific forms without departing from the spirit or essential attributes thereof and, accordingly, reference should be made to the appended claims, rather than to the foregoing specification, as indicating the scope of the invention.

1-15. (canceled)

16. A process for preparing a compound of formula ((S,S)-SDG-1), ((RR,)-SDG-2) or mixture of compounds of formula ((S,S)-SDG-1) and ((RR,)-SDG-2): 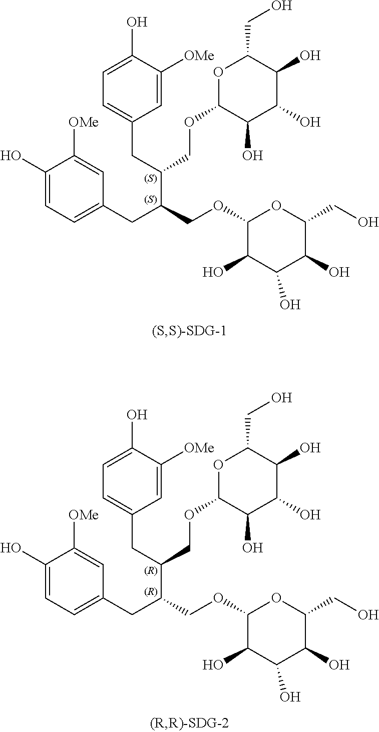
the process comprising:
(a) reacting a compound of formula (1a), (1b) or mixture of compounds of formula (1 a) and (1b) 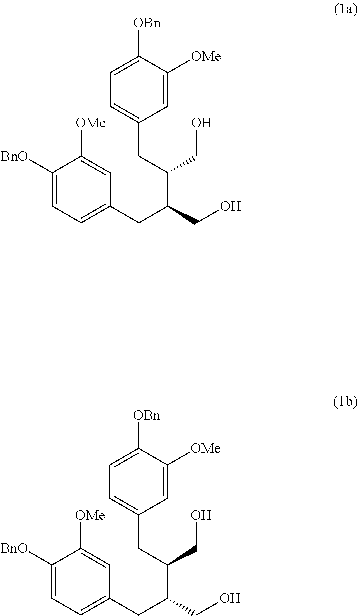 [SMILES: COc1cc(C[C@H](CO)[C@@H](CO)Cc2ccc(OCc3ccccc3)c(OC)c2)ccc1OCc1ccccc1 ; COc1cc(C[C@@H](CO)[C@H](CO)Cc2ccc(OCc3ccccc3)c(OC)c2)ccc1OCc1ccccc1]
with a compound of formula (2): 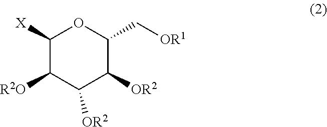
wherein X is a halogen, and R¹ and each R² are independently a protective group, to prepare a mixture of compounds of formula ((SS)-3) and ((R,R)-4): 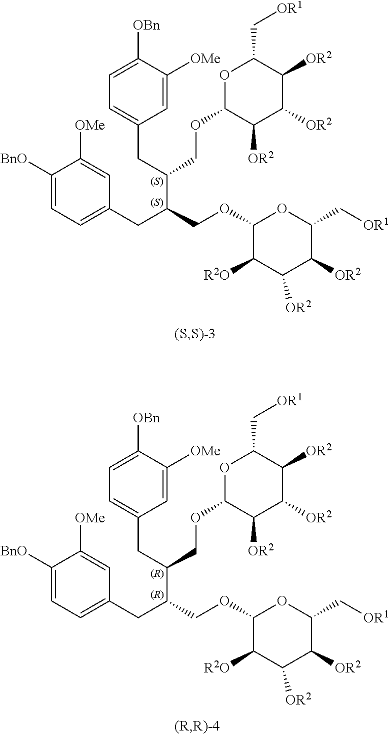
(b) cleaving benzyl ethers of the compounds of formula ((S,S)-3) and ((R,R)-4) to provide a mixture of compounds of formula ((S,S)-5) and ((R,R)-6): 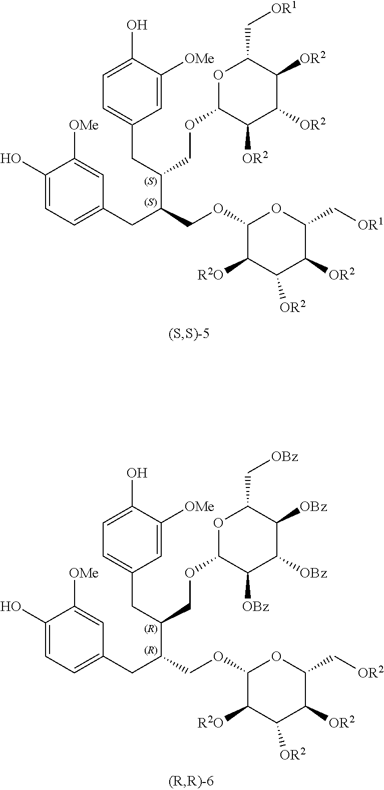
(c) optionally separating the mixture of compounds of formula ((S,S)-5) and formula ((R,R)-6) to provide a compound of formula ((S,S-5) or a compound of formula ((R,R)-6); and
(d) deprotecting the compound of formula ((S,S)-5), the compound of formula ((R,R)-6) or mixture of compounds of formula ((S,S-5) and (R,R)-6) to provide a compound of formula ((S,S)-SDG-1), a compound of formula ((R,R)-SDG-2) or mixture of compounds of formula ((S,S)-SDG-1) and ((R,R)-SDG-2).

17. A process for preparing a mixture of compounds of formula ((S,S)-3) and formula ((R,R)-4): 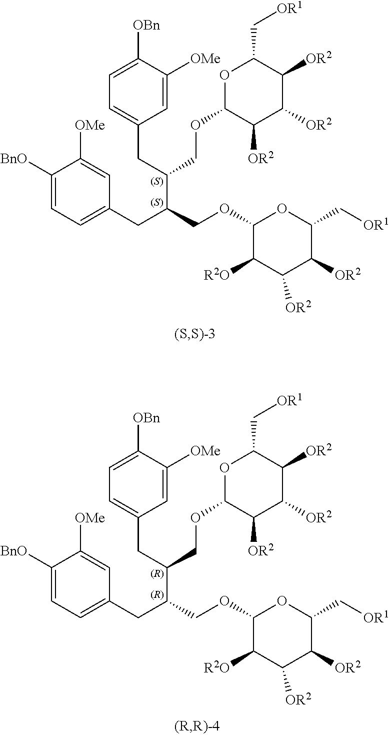
wherein R¹ and each R² are independently a protective group,
the process comprising:
reacting a compound of formula (1a), (1b), or mixture of compounds of formula (1a) and (1b): 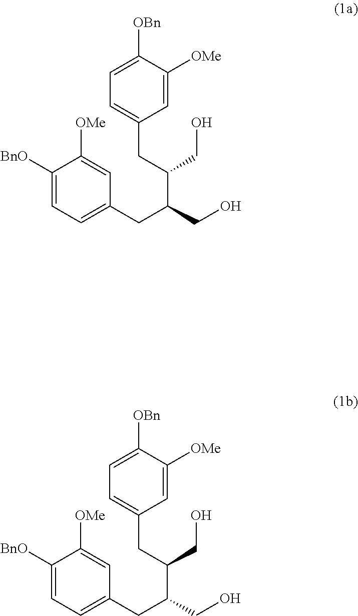 [SMILES: COc1cc(C[C@H](CO)[C@@H](CO)Cc2ccc(OCc3ccccc3)c(OC)c2)ccc1OCc1ccccc1 ; COc1cc(C[C@@H](CO)[C@H](CO)Cc2ccc(OCc3ccccc3)c(OC)c2)ccc1OCc1ccccc1]
with a compound of formula (2): 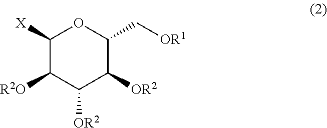
wherein X is a halogen, and R¹ and each R² are independently a protective group, to prepare a mixture of compounds of formula ((S,S-3) and formula ((R,R)-4).

18. A process for preparing a mixture of compounds of formula ((S,S)-5) and ((R,R)6): 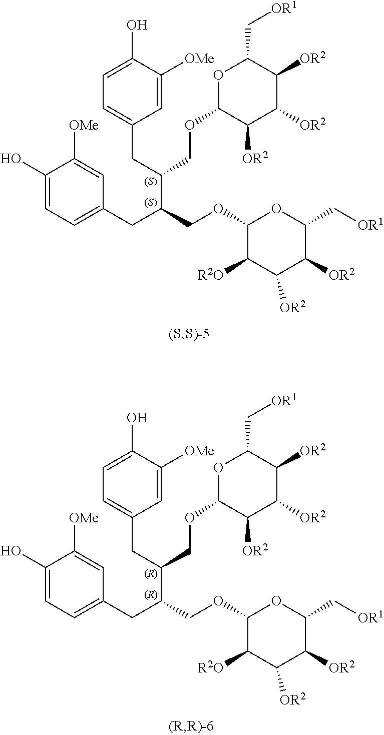
wherein R¹ and each R² are independently a protective group,
the process comprising:
(a) reading a compound of formula 1a), (1b), or mixture of compounds of formula (1a) and (1b): 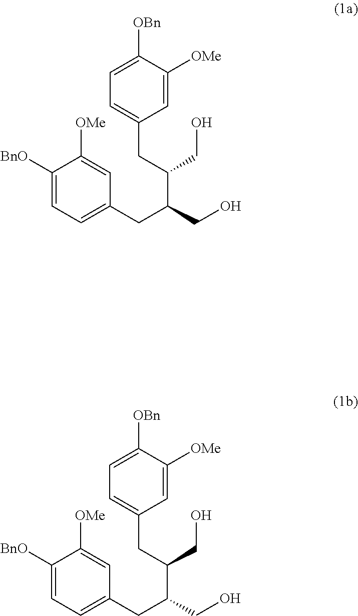 [SMILES: COc1cc(C[C@H](CO)[C@@H](CO)Cc2ccc(OCc3ccccc3)c(OC)c2)ccc1OCc1ccccc1 ; COc1cc(C[C@@H](CO)[C@H](CO)Cc2ccc(OCc3ccccc3)c(OC)c2)ccc1OCc1ccccc1]
with a compound of formula (2): 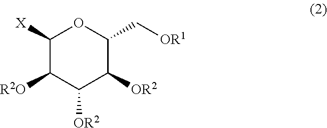
wherein X is a halogen, and R¹ and each R² are independently a protective group, to prepare a mixture of compounds of formula ((S,S)-3) and formula ((R,R)-4): 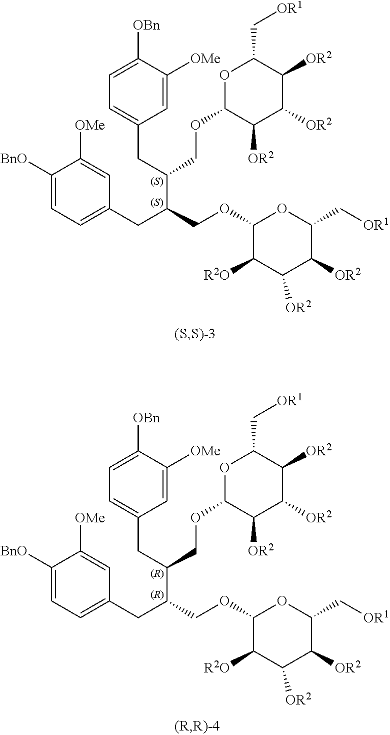
(b) cleaving benzyl ethers of the mixture of compounds of formula ((S,S)-3) and formula ((R,R)-4) to provide a mixture of compounds of formula ((S,S)-5) and ((R,R)-6).

19. The process according to claim 16, wherein the compound of formula (1a) is reacted in (a).

20. The process according to claim 16, wherein the compound of formula (1b) is reacted in (a).

21. The process according to claim 16 wherein a mixture of the compound of formula (1a) and the compound of formula (1b) is reacted in step (a).

22. The process according to claim 16, wherein a mixture of compounds of formula ((S,S)-5) and formula ((R,R)-6) is separated, and at least one of the compound of formula ((S,S)-5) and the compound of formula ((R,R)-6) is deprotected in step (d).

23. The process according to claim 16 wherein R¹ and each R² are independently selected from acetyl and benzoyl.

24. The process according to claim 23 wherein R¹ and each R² are benzoyl.

25. The process according to claim 16 wherein X in the compound of formula (2) is bromine.

26. The process according to claim 16 where step (a) is carried in the presence of a halide ion acceptor.

27. The process according to claim 16 where the halide ion acceptor is a heavy metal salt.

28. The process according to claim 27 wherein the heavy metal salt is a salt of silver or a salt of mercury.

29. The process according to claim 28 wherein the heavy metal salt is selected from the group consisting of CAgF₃O, Ag₂O, Ag₂CO₃, AgO₂CCH₃, AgClO₄, Hg(CN)₂, HgBr₂, and combinations thereof.

30. The process according to claim 16 where step (a) is carried in the presence of activated molecular sieves.
